(19) United States
(12) Patent Application Publication
(10) Pub. No.: US 2015/0202224 A1
(43) Pub. Date: Jul. 23, 2015

(54) METHOD OF TREATING IRON DEFICIENCY ANEMIA

(71) Applicant: Luitpold Pharmaceuticals, Inc., Shirley, NY (US)

(72) Inventors: David B. Bregman, Clarksburg, NJ (US); Marc L. Tokars, Douglassville, PA (US)

(21) Appl. No.: 14/417,369

(22) PCT Filed: Jul. 26, 2013
(86) PCT No.: PCT/US13/52299

§ 371 Date: Jan. 26, 2015

Related U.S. Application Data
(60) Provisional application No. 61/676,813, filed on Jul. 27, 2012.

(51) Int. Cl. A61K 31/7135; A61K 31/295; G01N 33/74; A61K 33/26

(52) U.S. Cl. CPC A61K 31/7135; A61K 33/26; A61K 31/295; G01N 33/74; G01N 2800/52; G01N 2800/22

(57) ABSTRACT

Methods for predicting responsiveness of a subject to iron therapy and treatment methods related thereto. Such methods can be useful with respect to conditions, disorders, or diseases associated with iron deficiency anemia.

### CROSS-REFERENCE TO RELATED APPLICATIONS

The present application claims the benefit of U.S. Provisional Application Ser. No. 61/676,813 filed 27 Jul. 2012, which is incorporated herein by reference in its entirety.

### FIELD OF THE INVENTION

The present disclosure generally relates to methods of treating iron deficiency related conditions and diagnostic biomarkers for use therewith.

### BACKGROUND OF THE INVENTION

Worldwide, iron deficiency anemia (IDA) is the most common nutritional deficiency (Clark, 2008). The etiology of IDA may result from inadequate intake of iron, impaired absorption, or losses of iron from various conditions (e.g., menstrual or gastrointestinal blood loss). Treatment options for IDA include supplementation with oral iron or intravenous iron therapy. Although oral iron has been the initial choice to treat subjects with IDA, there are subjects who do not tolerate the side effects of oral iron or who fail to produce an optimal response for management of IDA within a prescribed time period. In many subjects, inflammation or other co-existing conditions inhibit the ability of iron to be absorbed from the gastrointestinal tract or released effectively from storage iron to hematopoietic precursors (Weiss, 2005). Parenteral administration of iron has been used in these subjects when oral iron is not a feasible option.

The homeostasis of iron in the body is regulated in part by a peptide hormone called hepcidin, which is secreted by hepatocytes in the liver (Weiss, 2005). The action of hepcidin works upon a transmembrane iron efflux transporter called ferroportin (Coyne, 2011). This transporter is present on the basolateralsurface of enterocytes, and is also present in macrophages, hepatocytes, and placental cells (Nemeth, 2004; Ganz, 2006). The cell surface expression of ferroportin permits the movement of intracellular iron into plasma (Ganz, 2011). When hepcidin is bound to ferroportin, this transporter is internalized and degraded (Nemeth, 2004). Consequently, ferroportin can down regulate intestinal iron absorption as well as the release of iron from macrophages and iron stores within hepatocytes (Nemeth, 2004).

Absolute iron deficiency has traditionally been characterized by low serum iron, low percent transferrin saturation, and low ferritin (Goodnough, 2010). But laboratory results can be less sensitive or less specific in subjects who have co-morbid disease with inflammation, particularly when iron deficiency co-exists with inflammatory conditions leading to iron sequestration and hypoferremia (Freireich, 1957). Entrapment of iron can occur within macrophages and within gastrointestinal (GI) epithelial cells, impairing GI absorption. This re-distribution of iron during inflammation is thought to be due to cytokine-stimulated over production of hepcidin (Ganz, 2011). The identification of iron-restricted erythropoiesis due to absolute iron deficiency or iron sequestration can play a role in management of anemia (Goodnough, 2011).

Ferritin is a ubiquitous intracellular protein that stores iron and releases it in a controlled fashion. Ferritin serves to store iron in a non-toxic form, to deposit it in a safe form, and to transport it to areas where it is required. The amount of ferritin stored reflects the amount of iron stored. The protein is produced by almost all living organisms, including algae, bacteria, higher plants, and animals. In humans, it acts as a buffer against iron deficiency and iron overload. Ferritin is a globular protein complex consisting of 24 protein subunits and has been known as the primary intracellular iron-storage protein in both prokaryotes and eukaryotes, keeping iron in a soluble and non-toxic form. Ferritin that is not combined with iron is called apoferritin.

Serum ferritin levels are measured in medical laboratories as part of the iron panel for anemia and for restless legs syndrome. The ferritin levels measured usually have a direct correlation with the total amount of iron stored in the body. But ferritin levels may be artificially high in cases of anemia of chronic disease where ferritin is elevated in its capacity as an acute phase protein and not as a marker for iron overload. If the ferritin level is low, there is a risk for lack of iron, which could lead to anemia.

Hepcidin is a peptide hormone produced by the liver implicated in iron homeostasis. It was discovered in 2000, and is thought to be a regulator of iron homeostasis in humans and other mammals. Hepcidin functions to regulate iron transport across the gut mucosa, thereby preventing excess iron absorption and maintaining normal iron levels within the body.

Hepcidin inhibits iron transport by binding to the iron channel ferroportin, which is located on the basolateral surface of gut enterocytes and the plasma membrane of reticuloendothelial cells (macrophages). Inhibiting ferroportin shuts off the iron transport out of these cells, which store iron. By inhibiting ferroportin, hepcidin prevents enterocytes of the intestines from secreting iron into the hepatic portal system, thereby functionally reducing iron absorption. The iron release from macrophages is also prevented by ferroportin inhibition; therefore, the hepcidin maintains iron homeostasis. Correlation has been shown between hepcidin and serum ferritin (Wish, 2006 and Ganz, 2008). It was also recently thought that findings of lower hepcidin values are a result of lower iron stores (Ganz, 2008).

Hepcidin levels can be influenced by a number of factors. For example, expression and production of hepcidin can be regulated by iron status, inflammation, erythropoiesis, or oxygen tension (Fleming, 2012). As another example, increased plasma iron and iron stores are thought to stimulate hepcidin production, which ultimately can inhibit dietary iron absorption or decrease iron turnover. As another example, hepcidin expression can be stimulated by interleukin-6, an inflammatory cytokine, while increased erythropoetic activity (e.g. administration of erythropoietin, phlebotomy) can suppress hepcidin levels. As another example, hypoxia can exert an inhibitory effect on hepcidin production, mainly by hypoxia-inducible factor (HIF) (Fleming, 2012).

### SUMMARY OF THE INVENTION

Among the various aspects of the present disclosure is the provision of a method for treating iron deficiency with iron therapy tailored to subject based on the best predicted treatment outcome.

One aspect provides a method of determining response to iron therapy for treatment of iron deficiency anemia or a disease, disorder, or condition associated with iron deficiency anemia in a subject. In some embodiments, this method includes determining a level of hepcidin in a biological sample of a subject in need of iron therapy; and correlating a level of hepcidin equal to or greater than a predetermined hepcidin level with reduced responsiveness of the subject to oral iron therapy, or correlating a level of hepcidin less than the predetermined hepcidin level with at least adequate responsiveness of the subject to oral iron therapy.

Another aspect provides a method of treating iron deficiency anemia or a disease, disorder, or condition associated with iron deficiency anemia. In some embodiments, this method includes determining a level of hepcidin in a biological sample of a subject in need of iron therapy; and correlating a level of hepcidin equal to or greater than a predetermined hepcidin level with reduced responsiveness of the subject to oral iron therapy, or correlating a level of hepcidin less than the predetermined hepcidin level with at least adequate responsiveness of the subject to oral iron therapy. In some embodiments, this method includes administering a composition including iron, wherein administering the composition includes intravenously administering a first iron composition to the subject if the level of hepcidin is equal to or greater than the predetermined hepcidin level, or orally administering a second iron composition to the subject if the level of hepcidin is less than the predetermined hepcidin level.

Further embodiments of these various aspects are discussed below.

In some embodiments, the first iron composition includes an iron carbohydrate complex. In some embodiments, the first iron composition includes one or more of an iron carboxymaltose, iron dextran; sodium ferric gluconate complex in sucrose; ferumoxytol, iron sucrose; iron gluconate; iron dextrin; polymaltose; iron sucrose; iron saccharate complex; iron pyrophosphate; or iron sorbitol. In some embodiments, the first iron carbohydrate complex includes an iron carboxymaltose. In some embodiments, the second iron composition includes one or more of an iron (II) sulfate; ferrous sulfate; ferrous fumarate; heme iron polypeptide; ferrous glycine sulfate; iron pyrophosphate; or an iron carbohydrate complex.

In some embodiments, the disease, disorder, or condition associated with iron deficiency anemia includes at least one of: chronic blood loss; acute blood loss; pregnancy; a post-partum time period; a peripartum time period; dysfunctional uterine bleeding; heavy uterine bleeding; chronic recurrent hemoptysis; chronic internal bleeding; gastrointestinal bleeding; parasitic infections; chronic kidney disease; dialysis; surgery or acute trauma; use of erythropoiesis stimulating agents; insufficient dietary intake and absorption of iron; iron loss from intestinal bleeding; parasitic worms; bleeding ulcer; gastric ulcers; duodenal ulcers; gastrointestinal cancer; colon polyp; urinary tract bleeding; blood loss from injury, surgery, or frequent blood drawing; gastric bypass; disease of the intestine; Crohn's disease; celiac disease; malabsorption; or iron deficiency anemia of unknown etiology. In some embodiments, the iron deficiency anemia or the disease, disorder, or condition associated with iron deficiency anemia includes inflammation.

In some embodiments, reduced responsiveness of the subject to oral iron therapy includes less than about 1 g/dL increase in hemoglobin over about two weeks of oral iron treatment. In some embodiments, the at least adequate responsiveness of the subject to oral iron therapy includes more than about 1 g/dL increase in hemoglobin over about two weeks of oral iron treatment.

In some embodiments, determining a level of hepcidin in a subject includes a hepcidin immunoassay or a hepcidin mass spectrophotometry assay. In some embodiments, the predetermined hepcidin level is about 10 ng/mL, about 11 ng/mL, about 12 ng/mL, about 13 ng/mL, about 14 ng/mL, about 15 ng/mL, about 16 ng/mL, about 20 ng/mL, about 21 ng/mL, about 22 ng/mL, about 23 ng/mL, about 24 ng/mL, about 25 ng/mL, about 30 ng/mL, about 40 ng/mL, about 50 ng/mL, about 60 ng/mL, about 70 ng/mL, about 80 ng/mL, about 90 ng/mL, about 100 ng/mL, about 110 ng/mL, about 120 ng/mL, about 130 ng/mL, about 140 ng/mL, about 150 ng/mL, about 160 ng/mL, about 170 ng/mL, about 180 ng/mL, about 190 ng/mL, about 200 ng/mL, about 210 ng/mL, about 220 ng/mL, about 230 ng/mL, about 240 ng/mL, about 250 ng/mL, about 260 ng/mL, about 270 ng/mL, about 280 ng/mL, about 290 ng/mL, about 300 ng/mL, about 310 ng/mL, about 320 ng/mL, about 330 ng/mL, about 340 ng/mL, about 350 ng/mL, about 360 ng/mL, or about 370 ng/mL. In some embodiments, the predetermined hepcidin level is about 10 ng/ml. In some embodiments, the predetermined hepcidin level is about 15 ng/ml. In some embodiments, the predetermined hepcidin level is about 20 ng/ml.

In some embodiments, the subject is a mammal. In some embodiments, the subject is a human. In some embodiments, the biological sample includes at least one selected from the group consisting of urine, whole blood, or a blood component. In some embodiments, the biological sample includes serum.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1 is a flow chart depicting subject selection procedure. Subjects were eligible if they exhibited hemoglobin levels ≦11 g/dL with IDA of any etiology and satisfied the other inclusion and exclusion criteria. Screening hepcidin levels were measured in 240 subjects prior to the oral iron run-in (Day 15). If subjects had a <1 g/dL hemoglobin increase at the end of the run-in (non-responders), they were randomized to FCM or oral iron. Further details regarding methodology are provided in Example 1.

FIG. 2 is a scatter plot of Screening Hemoglobin (y-axis) vs. Hepcidin (x-axis) with Response to Oral Iron Identified. FIG. 2 shows hemoglobin level and respective hepcidin level for an initial forty-four subjects after a 14 day oral iron run-in. Subjects with “Y” were defined as responders and subjects with “N” were defined as non-responders. Response was defined as a hemoglobin increase of >1 g/dL after run-in. Further details regarding methodology are provided in Example 2 and Example 4.

FIG. 3 is a Receiver Operating Characteristic (ROC) curve for Sensitivity vs. 1-Specificity for hepcidin, ferritin, and TSAT. Various cutoff levels are indicated near the respective curves in the same color as the respective curves (see e.g., Table 6, Table 7, and Table 8, respectively). The ROC curves demonstrate the ability or inability of baseline hepcidin, ferritin or TSAT to predict response to oral iron in IDA subjects. Further details regarding methodology are provided in Example 6.

### DETAILED DESCRIPTION OF THE INVENTION

The present disclosure is based, at least in part, on the discovery that hepcidin can be used as a biomarker for clinical assessment of subjects with iron deficiency anemia (IDA). Studies described herein identified a hepcidin screening level of 20 ng/mL as an accurate predictor of subject response to iron therapy. Also shown herein, hepcidin was a more effective biomarker for clinical assessment of IDA subjects than ferritin values, which is a poor predictor of subject response.

As shown herein, hepcidin levels can be a more effective biomarker predictive of response to iron therapy as compared to other molecules associated with iron metabolism, such as TSAT or ferritin. Hepcidin, TSAT, and ferritin are understood to be acute phase reactants. TSAT and ferritin have been described as less than ideal tests because of their acute phase reactivity status. While ferritin levels have been shown to be correlated with hepcidin levels (see e.g., Example 5; see generally Wish, 2006), it is not an ideal test given that it is an acute phase reactant. Similarly, TSAT is a less than ideal tests because of its acute phase reactivity status. Thus, the discovery that hepcidin, itself an acute phase reactant, is a more effective biomarker than TSAT or ferritin was unexpected. The present disclosure provides a diagnostic method for predicting subject responsiveness to IV iron therapy versus oral iron therapy that is superior to conventional approaches. Results comparing IV iron to oral iron in chronic kidney disease (CKD) subjects have been contradictory, but more recent evidence shows IV treatment to be more effective at achieving a recommended hemoglobin level (Van Wyck, 2005).

Among the various aspects described herein is a method for predicting a subject's responsiveness to iron therapy. Such method can be used as a diagnostic criterion for administration of, for example, either an intravenous iron formulation or an oral iron formulation. Thus is provided increased efficacy of therapeutic administration of iron for the treatment of, for example, a disease, disorder, or condition associated with iron deficiency anemia. As described more fully below, a level of hepcidin can be determined in a subject (e.g., a subject in need or thought to be in need of iron therapy). Based on a determined level of hepcidin, one can predict whether the subject will respond more favorable to administration of an oral iron formulation or an intravenous iron formulation. For example, a level of 20 ng/mL or greater can be the basis for predicting the subject will respond more favorable to administration of an intravenous iron formulation compared to an oral iron formulation. Based on a determined level of hepcidin, a subject can be administered an oral iron formulation or an intravenous iron formulation. For example, based on determination of a hepcidin level of 20 ng/mL or greater, an intravenous iron formulation can be administered to a subject in need of or thought to be in need of iron therapy instead of or in addition to administration of oral iron.

Iron Deficiency Anemia and Diseases, Disorders, or Conditions Associated Therewith

A disease, disorder, or condition associated with iron deficiency anemia can be treated via iron supplementation. Provided herein is a method of predicting a subject's responsiveness to various forms of iron therapy as well as therapeutic approaches that can be used with such predictive methods. A subject can be in need of iron therapy are those with iron deficiency anemia.

Iron deficiency anemia, and diseases, disorders, or conditions associated with iron deficiency anemia such, are well-known (see e.g., Clark, 2008). Therapeutic treatment protocols for iron deficiency anemia (e.g., administration of an oral iron supplement or intravenous iron carbohydrate complex) are well-known (see e.g., Van Wyck et al. (2004) J Am Soc Nephrol 15, S91-S92). Assays for detecting or diagnosing a disease, disorder, or condition associated with iron deficiency anemia are well-known (see e.g., Aspuru, 2011; Zhu, 2010; Clark, 2008; Alleyne, 2008). Except as otherwise noted herein, therefore, the process of the present disclosure can be carried out in accordance with such conventional understanding of iron deficiency anemia and detection, diagnosis, or treatment thereof.

Exemplary diseases, disorders, or conditions associated with iron deficiency anemia include, but are not limited to: chronic blood loss; acute blood loss; pregnancy; childbirth; childhood development; psychomotor and cognitive development in children; breath holding spells; dysfunctional uterine bleeding; heavy uterine bleeding; menstruation; chronic recurrent hemoptysis; idiopathic pulmonary siderosis; chronic internal bleeding; gastrointestinal bleeding; parasitic infections; chronic kidney disease; dialysis; surgery or acute trauma; and chronic ingestion of alcohol, chronic ingestion of salicylates, chronic ingestion of steroids; chronic ingestion of non-steroidial anti-inflammatory agents, chronic ingestion of erythropoiesis stimulating agents; insufficient dietary intake and absorption of iron; iron loss from intestinal bleeding; parasitic worms (such as hookworms; whipworms; and roundworms); bleeding ulcer; gastric ulcers; duodenal ulcers; gastrointestinal cancer; colon polyp; deficient levels of hemoglobin; use of proton pump inhibitors; use of antacids; urinary tract bleeding; blood loss from injury, surgery, or frequent blood drawing; gastric bypass; disease of the intestine; Crohn's disease; celiac disease; or malabsorption. As used herein, a disease, disorder, or condition associated with iron deficiency anemia can include iron deficiency anemia per se.

Anemia of chronic disease is associated with, for example, rheumatoid arthritis; cancer; Hodgkins leukemia; non-Hodgkins leukemia; cancer chemotherapy; inflammatory bowel disease; ulcerative colitis thyroiditis; hepatitis; systemic lupus erythematosus; polymyalgia rheumatica; scleroderma; mixed connective tissue disease; Sojgren's syndrome; congestive heart failure/cardiomyopathy; and idiopathic geriatric anemia.

Anemia can also be associated with, for example, Crohn's Disease; gastric surgery; ingestion of drug products that inhibit iron absorption; and chronic use of calcium.

States characterized by dysfunctional iron metabolism and treatable with compositions, predictive methods, and therapeutic methods described herein include, but are not limited to, restless leg syndrome; blood donation; Parkinson's disease; hair loss; and attention deficit disorder.

Hemoglobin is the iron-containing oxygen-transport metalloprotein in red blood cells. Anemia can be characterized by a decrease in hemoglobin. Decrease of hemoglobin, with or without an absolute decrease of red blood cells, can lead to symptoms of anemia. Anemia can have many different causes, with iron deficiency and resultant iron deficiency anemia being the most common.

IDA is a common anemia (low red blood cell level) can be caused by insufficient dietary intake and absorption of iron, and/or iron loss from intestinal bleeding. Red blood cells contain iron and are not formed when iron is deficient. Iron deficiency causes approximately half of all anemia cases worldwide, and affects women more often than men. World estimates of iron deficiency occurrence are somewhat vague, but the true number probably exceeds one billion persons. The most significant cause of iron-deficiency anemia is parasitic worms, such as hookworms; whipworms; and roundworms. Malaria, hookworms and vitamin A deficiency contribute to anemia during pregnancy in most underdeveloped countries. In women over 50 years old the most common cause of iron-deficiency anemia is chronic gastrointestinal bleeding from nonparasitic causes, such as gastric ulcers, duodenal ulcers, or gastrointestinal cancer. In premenopausal women, menstrual blood loss and pregnancy-related iron losses account for most IDA diagnoses. Nevertheless, occult bleeding from the gastrointestinal (GI) tract is the leading cause for IDA in men and postmenopausal women. Bleeding lesions in the GI tract are identified in about 50% of subjects with IDA. GI malignancy is also significantly more common in men and postmenopausal women with IDA. About one-third of the subjects with inflammatory bowel disease (IBD) are anemic (Zhu, 2010).

Anemia is one result of advanced-stage iron deficiency. When the body has sufficient iron to meet its needs (functional iron), the remainder is stored for later use in all cells, but mostly in the bone marrow, liver, and spleen. These stores are called ferritin complexes and are part of the human (and other animals) iron metabolic systems. Ferritin complexes in humans carry approximately 4500 iron atoms and form into 24 protein subunits of two different types.

Ferritin level has been conventionally used (along with transferrin saturation values) as a diagnostic tool to identify iron deficiency. In the setting of anemia, low serum ferritin is thought to be the most specific lab test for IDA. Serum ferritin, however, is less sensitive, since levels are increased in the blood by infection or any type of chronic inflammation, and these conditions may convert what would otherwise be a low level of ferritin from lack of iron, into a value in the normal range. It has also been shown that hepcidin and serum ferritin respond similarly to inflammation and changes in iron stores, and is reflected in the strong correlation between hepcidin and ferritin in healthy volunteers (Ganz, 2008).

TSAT is the ratio of serum iron and total iron-binding capacity, multiplied by 100. Of the transferrin that is available to bind iron, the TSAT value tells a clinician how much serum iron is actually bound. For example, a value of 15% means that 15% of iron-binding sites of transferrin is being occupied by iron. Results of TSAT and ferritin are usually reported together.

Iron-deficiency anemia is characterized by the sign of pallor (reduced oxyhemoglobin in skin or mucous membranes), and the symptoms of fatigue, lightheadedness, and weakness. None of the symptoms (or any of the others below) are sensitive or specific. Pallor of mucus membranes (primarily the conjunctiva in children is the sign of anemia with best correlation to the actual disease, but in a large study was found to be only 28% sensitive and 87% specific (with high predictive value) in distinguishing children with anemia (Hb<11.0 g/dL) and 49% sensitive and 79% specific in distinguishing severe anemia (Hb<7.0 g/dL). Thus, this sign is reasonably predictive when present, but not helpful when absent, as only one-third to one-half of children who are anemic (depending on severity) will show pallor. Iron-deficiency can be diagnosed by laboratory testing.

Because iron deficiency tends to develop slowly, adaptation occurs and the disease often goes unrecognized for some time. In severe cases, dyspnea (trouble breathing) can occur. Unusual obsessive food cravings, known as pica, may develop. Pagophagia or pica for ice has been suggested to be specific, but is actually neither a specific or sensitive symptom, and is not helpful in diagnosis. When present, it may (or may not) disappear with correction of iron-deficiency anemia.

Other symptoms and signs of iron deficiency anemia can include:

Restless Legs Syndrome; anxiety; Obsessive Compulsive Disorder (OCD); irritability; angina; constipation; sleepiness; tinnitus; mouth ulcers; palpitations; hair loss; fainting; faintness; depression; breathlessness; twitching muscles; pale yellow skin; tingling, numbness, or burning sensations; missed menstrual cycle; heavy menstrual period; slow social development; glossitis; angular cheilitis; koilonychia; poor appetite; pruritus; dysphagia; and insomnia.

### Hepcidin and Detection Thereof

As described herein, hepcidin levels can be used to predict responsiveness to iron therapy. Studies showed that hepcidin levels were correlated with reduced responsiveness to oral iron therapy (see e.g., Example 6). By comparing the hepcidin level of a biological sample of a subject to a predetermined level of hepcidin, one can predict responsiveness of the subject to various forms of iron therapy. For example, a value 20 ng/mL or greater of hepcidin can indicate an increased responsiveness of the subject to intravenous iron therapy and a value less than a 20 ng/mL of hepcidin can indicate an increased responsiveness of the subject to oral iron therapy.

Assays (e.g., an immunoassay or a mass spectrophotometry assay) for determination of levels of hepcidin in a subject sample (e.g., serum hepcidin) are well known (see e.g., Ganz, 2008). Except as otherwise noted herein, therefore, the process of the present disclosure can be carried out in accordance with such conventional understanding of hepcidin assays. Hepcidin levels in a biological sample can be measured as a bioactive, circulating form of hepcidin, such as a 25 amino acid isoform (i.e., hepcidin-25), a 22 amino acid isoform (i.e., hepcidin-22), or a 20 amino acid isoform (i.e., hepcidin-20). Because hepcidin-25 is understood to play a role in iron metabolism, in various embodiments, levels of hepcidin-25 can be determined.

Hepcidin levels can be determined from a variety of biological samples of a subject. For example, hepcidin levels can be determined from a urine sample of a subject.

As another example, hepcidin levels can be determined from whole blood sample of a subject. As another example, hepcidin levels can be determined from blood component sample of a subject. Whole blood generally comprises plasma, serum, and blood cells. Blood components include, but are not limited to, red blood cells, white blood cells (e.g., leukocytes or platelets, i.e., thrombocytes), plasma, serum, hemoglobin, water, proteins, glucose, amino acids, fatty acids, mineral ions, hormones, carbon dioxide, urea, and lactic acid.

As another example, hepcidin levels can be determined from a plasma sample of a subject. Blood plasma can include one or more of water, proteins, glucose, amino acids, fatty acids, mineral ions, hormones, carbon dioxide, urea, lactic acid, platelets (i.e., thrombocytes), and blood cells. In a human subject, blood plasma represents about 55% of whole blood, or about 2.7 to 3 liters in an average human subject. Blood plasma contains about 92% water, 8% blood plasma proteins, and trace amounts of other materials. Blood plasma can contain serum albumin, blood-clotting factors, immunoglobulins, lipoproteins, other proteins, and electrolytes (e.g., sodium and chloride). A crude sample comprising blood plasma can also contain blood cells.

As another example, hepcidin levels can be determined from a serum sample of a subject. Blood serum is generally understood as plasma from which clotting proteins have been removed, leaving mostly albumin and immunoglobulins. Hepcidin threshold levels discussed below are based on serum sample levels.

It is thought that hepcidin baseline levels can vary between different sample types (e.g., serum versus urine). One of ordinary skill will understand that threshold values based on hepcidin levels in serum as discussed below can be extrapolated to other sample types (e.g., urine) based upon relative baseline and departure therefrom.

Various embodiments of methods described herein involve determining a level of hepcidin in a subject. Based on a determined level of hepcidin, one can predict whether the subject will respond more favorable to administration of an oral iron formulation or an intravenous iron formulation. Based on a determined level of hepcidin, a subject can be administered an oral iron formulation or an intravenous iron formulation.

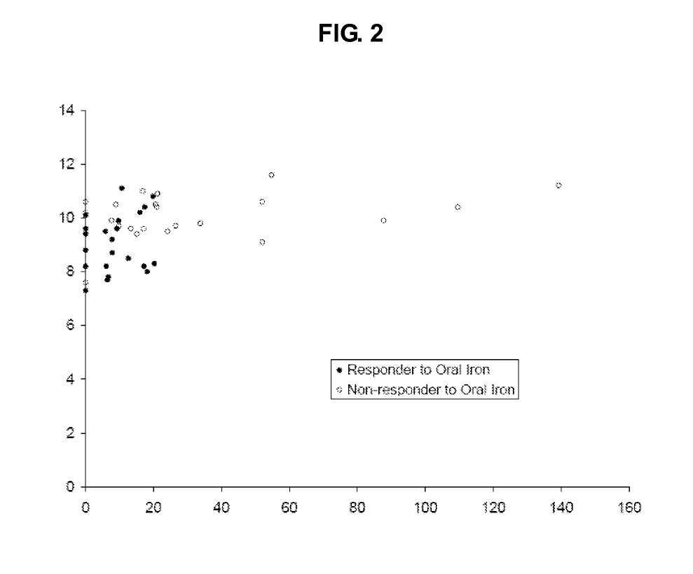

Studies described herein show that hepcidin levels in serum above 10 ng/mL provide a positive predictive value (PPV) of approximately 60%; hepcidin levels in serum above 15 ng/mL provide a PPV of approximately 70%; and hepcidin levels in serum above 20 ng/mL provide a PPV of 82% (see e.g., FIG. 2; Example 4; Example 6). Such hepcidin PPV values provide superior results compared to ferritin or TSAT (59% and 55% PPV, respectively) (see e.g., Example 6).

A predictive threshold of hepcidin can be used to predict responsiveness of a subject to iron therapy, where, for example, hepcidin above the selected threshold indicates adequate response to oral iron and hepcidin below the selected threshold indicates reduced responsiveness to oral iron or increased responsiveness to intravenous iron. In some embodiments, a predictive threshold of hepcidin in serum is at least about 1 ng/mL. For example, a predictive threshold of hepcidin in serum can be at least about 2 ng/mL, at least about 3 ng/mL, at least about 4 ng/mL, at least about 5 ng/mL, at least about 6 ng/mL, at least about 7 ng/mL, at least about 8 ng/mL, at least about 9 ng/mL, at least about 10 ng/mL, at least about 11 ng/mL, at least about 12 ng/mL, at least about 13 ng/mL, at least about 14 ng/mL, at least about 15 ng/mL, at least about 16 ng/mL, at least about 17 ng/mL, at least about 18 ng/mL, at least about 19 ng/mL, at least about 20 ng/mL, at least about 21 ng/mL, at least about 22 ng/mL, at least about 23 ng/mL, at least about 24 ng/mL, at least about 25 ng/mL, at least about 30 ng/mL, at least about 35 ng/mL, at least about 40 ng/mL, at least about 45 ng/mL, at least about 50 ng/mL, at least about 55 ng/mL, at least about 60 ng/mL, at least about 70 ng/mL, at least about 80 ng/mL, at least about 90 ng/mL, at least about 100 ng/mL, at least about 110 ng/mL, at least about 120 ng/mL, at least about 130 ng/mL, at least about 140 ng/mL, at least about 150 ng/mL, at least about 160 ng/mL, at least about 170 ng/mL, at least about 180 ng/mL, at least about 190 ng/mL, at least about 200 ng/mL, at least about 210 ng/mL, at least about 220 ng/mL, at least about 230 ng/mL, at least about 240 ng/mL, at least about 250 ng/mL, at least about 260 ng/mL, at least about 270 ng/mL, at least about 280 ng/mL, at least about 290 ng/mL, at least about 300 ng/mL, at least about 310 ng/mL, at least about 320 ng/mL, at least about 330 ng/mL, at least about 340 ng/mL, at least about 350 ng/mL, at least about 360 ng/mL, or at least about 370 ng/mL.

A hepcidin level of a subject of 10 ng/mL or greater can be used to predict that a subject will have reduced responsiveness to oral iron therapy and therefore would be more appropriately treated with intravenous iron therapy without initially administering oral iron therapy. A hepcidin level of a subject of less than 10 ng/mL can be used to predict that a subject will be responsive to oral iron therapy.

A hepcidin level of a subject of 15 ng/mL or greater can be used to predict that a subject will have reduced responsiveness to oral iron therapy and therefore would be more appropriately treated with intravenous iron therapy without initially administering oral iron therapy. A hepcidin level of a subject of less than 15 ng/mL can be used to predict that a subject will be responsive to oral iron therapy.

A hepcidin level of a subject of 20 ng/mL or greater can be used to predict that a subject will have reduced responsiveness to oral iron therapy and therefore would be more appropriately treated with intravenous iron therapy without initially administering oral iron therapy. A hepcidin level of a subject of less than 20 ng/mL can be used to predict that a subject will be responsive to oral iron therapy.

Reduced responsiveness of the subject to oral iron therapy can be characterized as less than an about 1 g/dL increase in hemoglobin over about two weeks of oral iron treatment. Similarly, responsiveness of the subject to oral iron therapy can be characterized as more than an about 1 g/dL increase in hemoglobin over about two weeks of oral iron treatment.

IV Iron

Various methods described herein can be used to predict a subject's responsiveness to administration of an intravenous iron formulation.

Therapeutic administration of intravenous formulations of iron is well known (see e.g., Allyene, 2008; Zhu, 2010) Except as otherwise noted herein, therefore, the processes of the present disclosure can be carried out in accordance with such conventional understanding of and therapeutic administration of intravenous formulations of iron.

As described herein, IV supplementation of iron can be used for treatment of Iron deficiency or Iron Deficiency Anemia (IDA). Intravenous (IV) iron supplementation is understood to be a method of delivering iron by injection (shot) with a needle, either through a muscle or into a vein. Medication that is given through an injection or intravenously is called parenteral therapy. Intravenous iron is delivered into the subject's vein through a needle.

Various iron compositions can be used in IV iron therapy (e.g., iron pyrophosphates, iron carbohydrate complexes). There are various preparations available for IV iron therapy such as iron pyrophosphate, iron dextran, iron sucrose, iron gluconate, iron dextrin (polymaltose), and ferric carboxymaltose (Zhu, 2010). Several examples of parenteral or IV iron preparations commercially available include INFeD® (iron dextran); Dexferrum® (iron dextran); Ferrlecit® (Na ferric gluconate complex); Feraheme® (ferumoxytol); Venofer® (iron sucrose or iron saccharate complex); and Jectofer® (iron sorbitol).

An iron carbohydrate complex can be present in a variety of formulations discussed herein. For example, an intravenous iron formulation can include an iron carbohydrate complexes. Iron carbohydrate complex can also be present in an oral iron formulation.

Iron carbohydrate complexes are well known, commercially available, or have well known syntheses (see e.g., Andreasen and Christensen 2001, Geisser et al. 1992 Structure/histotoxicity relationship of parenteral iron preparations. Arzneimittelforschung 42: 1439-1452; Groman and Josephson 1990, Groman et al. 1989). Examples of iron carbohydrate complexes include, but are not limited to, iron monosaccharide complexes, iron disaccharide complexes, iron oligosaccharide complexes, and iron polysaccharide complexes, such as: iron carboxymaltose, iron sucrose, iron polyisomaltose, iron dextran, iron polymaltose, iron dextrin, iron gluconate, iron sorbitol, iron hydrogenated dextran, and ferumoxytol, which may be further complexed with other compounds, such as sorbitol, citric acid and gluconic acid (for example iron dextrin-sorbitol-citric acid complex and iron sucrose-gluconic acid complex), and mixtures thereof.

Parenteral iron formulations approved for use in the U.S. include iron dextran (e.g., InFed, Dexferrum), sodium ferric gluconate complex in sucrose (Ferrlecit), ferumoxytol (Feraheme), and iron sucrose (Venofer).

An iron carbohydrate complex can be as described in U.S. Pat. No. 6,960,571, issued 1 Nov. 2005; U.S. Pat. No. 7,754,702, issued 13 Jul. 2010; International Patent Application No. WO 2007/081744, published 19 Jul. 2007; US Patent Application Publication No. 2010/0266644, published 21 Oct. 2010.

An iron carbohydrate complex can be an iron carboxymaltose complex. An iron carboxymaltose complex can be according to U.S. Pat. No. 7,754,702, issued 13 Jul. 2010, or US Patent Application Publication No. 2010/0266644, published 21 Oct. 2010. An example of an iron carboxymaltose complex is polynuclear iron (III)-hydroxide 4(R)-(poly-(1→4)-O-α-glucopyranosyl)-oxy-2(R),3(S),5(R),6-tetrahydroxy-hexanoate (“FCM”). FCM is a Type I polynuclear iron (III) hydroxide carbohydrate complex that can be administered as parenteral iron replacement therapy for the treatment of various anemia-related conditions as well as other iron-metabolism related conditions. FCM can be represented by the chemical formula: [FeOx(OH)y(H2O)z]n [{(C6H10O5)m(C6H12O7)}l]k, where n is about 103, m is about 8, l is about 11, and k is about 4). The molecular weight of FCM is about 150,000 Da.

The degradation rate and physicochemical characteristics of FCM make it an efficient means of parenteral iron delivery to the body stores. It is more efficient and less toxic than the lower molecular weight complexes such as iron sorbitol/citrate complex, and does not have the same limitations of high pH and osmolarity that leads to dosage and administration rate limitations in the case of, for example, iron sucrose and iron gluconate.

FCM does not contain dextran and does not react with dextran antibodies; therefore, the risk of anaphylactoid/hypersensitivity reactions is very low compared to iron dextran. FCM has a nearly neutral pH (5.0 to 7.0) and physiological osmolarity, which makes it possible to administer higher single unit doses over shorter time periods than other iron-carbohydrate complexes. FCM can mimic physiologically occurring ferritin. FCM is metabolized by the glycolytic pathway. Like iron dextran, FCM is more stable than iron gluconate or sucrose. FCM produces a slow and competitive delivery of the complexed iron to endogenous iron binding sites resulting in an acute toxicity one-fifth that of iron sucrose.

After intravenous administration, FCM is mainly found in the liver, spleen, and bone marrow. Pharmacokinetic studies using positron emission tomography have demonstrated a fast initial elimination of radioactively labeled iron (Fe) ⁵²Fe/⁵⁹Fe FCM from the blood, with rapid transfer to the bone marrow and rapid deposition in the liver and spleen. See e.g., Beshara et al. (2003) Br J Haematol 2003; 120(5): 853-859. Eight hours after administration, 5 to 20% of the injected amount was observed to be still in the blood, compared with 2 to 13% for iron sucrose. The projected calculated terminal half-life (t½) was approximately 16 hours, compared to 3 to 4 days for iron dextran and 6 hours for iron sucrose.

Single-dose toxicity studies have demonstrated safety and tolerance in rodents and dogs of intravenous doses of FCM up to 60 times more than the equivalent of an intravenous infusion of 1,000 mg iron once weekly in humans. Pre-clinical studies in dogs and rats administered FCM in cumulative doses up to 117 mg iron/kg body weight over 13 weeks showed no observed adverse effect level in dose-related clinical signs of iron accumulation in the liver, spleen, and kidneys. No treatment-related local tissue irritation was observed in intra-arterial, perivenous, or intravenous tolerance studies in the rabbit. In vitro and in vivo mutagenicity tests provided no evidence that FCM is clastogenic, mutagenic, or causes chromosomal damage or bone marrow cell toxicity. There were no specific responses to FCM in a dextran antigenicity test.

Another iron carbohydrate complex for use in methods described herein is a polyglucose sorbitol carboxymethyl ether-coated non-stoichiometric magnetite (e.g., “ferumoxytol”). Ferumoxytol is known in the art to be effective for treating anemia (see e.g., Spinowitz et al. (2005) Kidney Intl 68, 1801-1807). Ferumoxytol is a superparamagnetic iron oxide that is coated with a low molecular weight semi-synthetic carbohydrate, polyglucose sorbitol carboxymethyl ether. Ferumoxytol and its synthesis are described in U.S. Pat. No. 6,599,498. Safety, efficacy, and pharmacokinetics of ferumoxytol are as described, for example, in Landry et al. (2005) Am J Nephrol 25, 400-410, 408; and Spinowitz et al. (2005) Kidney Intl 68, 1801-1807.

Oral Iron

Various methods described herein can be used to predict a subject's responsiveness to administration of an oral iron formulation.

Formulation and therapeutic administration of oral iron is well known (see e.g., Allyene, 2008; Zhu, 2010) Except as otherwise noted herein, therefore, the process of the present disclosure can be carried out in accordance with such conventional understanding of formulation and therapeutic administration of oral iron.

While oral supplementation of iron enjoys ease of use and convenience, oral iron supplementation is known to be poorly tolerated in some subjects. Furthermore, subjects may be non-compliant, not only from side effects of oral iron but because of the diagnosis or therapy they are undergoing for their disease.

Iron can be orally supplemented using various pharmacological forms, such as iron (II) sulfate, the most common and inexpensive salt (e.g. Feratab, Fer-Iron, Slow-FE, etc.) and in complex with gluconate, polysaccharide, dextran, fumurate, carbonyl iron, heme (e.g., feraheme), pyrophosphate, and other salts (see e.g., Allyene, 2008). Ascorbic acid may be added for enhanced absorption. Exemplary commercial oral iron formulations include, but are not limited to : Icar Pediatric; Fesol Caplets; Ircon; Hemocyte; Ferrous Fumarate Tablets; Nephro-Fen Feostat; Ferrous Fumarate with DSS Timed capsules; Ferro-DSS Caplets; Ferro-Sequels; Fergon; Ferrous Gluconate Tablets; Ferrous Sulfate Elixer; Ferrous Sulfate SolutionI Fer-Gen-Sol Drops; Mol-Iron; Feratab; Ferrous Sulfate Tablets EC; Ferrous Sulfate Tablets; Feosol; Slow FE; Ferres-150; Fe-Tinic; Hytinic; Niferes-150; Niferex-Elixer; Niferex; Enfamil Fer-In-Sol; EZFE 200; Fe-20; Femiron; Feosol; Ferate; Fer-Iron; Ferretts IPS; Ferrimin 150; Good Neighbor Pharmacy Iron; Good Neighbor Pharmacy Slow Release Iron; Leader Iron Tablets; Natural Slow Release Iron; Nature's Blend Slow Iron; ProFe; Proferrin ES; Right Aid Iron; Right Aid Slow Release Iron; or Slow Fe.

Heme iron polypeptide (HIP) can also be used as an oral iron when conventional iron supplements such as ferrous sulfate or ferrous fumarate are not tolerated or absorbed.

Another exemplary oral iron is ferrous glycine sulfate (or ferroglycine sulfate). Ferrous glycine sulfate may have less gastrointestinal side-effects than standard preparations such as iron fumarate, but is generally more expensive. Ferrous glycine sulfate can be useful in iron deficiency anemia associated with autoimmune gastritis or Heliobacter pylori gastritis.

Iron carbohydrates, as described in greater detail herein, can also be used as an oral iron.

Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005). Such formulations will contain a therapeutically effective amount of a biologically active agent described herein, which can be in purified form, together with a suitable amount of carrier so as to provide the form for proper administration to the subject.

As used herein, an iron agent can be any compound or composition comprising iron. For example, an iron agent can be an intravenous iron formulation (e.g., an intravenous iron carbohydrate complex) or an oral iron formulation.

The formulation should suit the mode of administration. The agents of use with the current disclosure can be formulated by known methods for administration to a subject using several routes which include, but are not limited to, parenteral, pulmonary, oral, topical, intradermal, intramuscular, intraperitoneal, intravenous, subcutaneous, intranasal, epidural, ophthalmic, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic or other physical forces.

A pharmaceutically acceptable carrier includes any and all solvents, dispersion media, coatings, antibacterial and anti-fungal agents, isotonic and absorption delaying agents, and the like, compatible with pharmaceutical administration (see e.g., Banker, Modern Pharmaceutics, Drugs and the Pharmaceutical Sciences, 4th ed. (2002) ISBN 0824706749; Remington The Science and Practice of Pharmacy, 21st ed. (2005) ISBN 0781746736). Preferred examples of such carriers or diluents include, but are not limited to, water, saline, Finger's solutions and dextrose solution. Supplementary active compounds can also be incorporated into the compositions. For intravenous administration, the iron carbohydrate complex is preferably diluted in normal saline to approximately 2-5 mg/mL. The volume of the pharmaceutical solution is based on the safe volume for the individual subject, as determined by a medical professional.

IV Iron.

In many cases, an iron carbohydrate complex can be delivered as a simple composition comprising the iron complex and the buffer in which it is dissolved. Other products can be added, if desired, for example, to maximize iron delivery, preservation, or to optimize a particular method of delivery.

An iron complex composition described herein can be formulated to be compatible with the intended route of administration, such as intravenous injection. Solutions and suspensions used for parenteral, intradermal or subcutaneous application can include a sterile diluent, such as water for injection, saline solution, polyethylene glycols, glycerine, propylene glycol or other synthetic solvents; antibacterial agents such as benzyl alcohol or methyl parabens; antioxidants such as ascorbic acid or sodium bisulfite; buffers such as acetates, citrates or phosphates, and agents for the adjustment of tonicity such as sodium chloride or dextrose. The pH can be adjusted with acids or bases, such as hydrochloric acid or sodium hydroxide. Preparations can be enclosed in ampules, disposable syringes or multiple dose vials made of glass or plastic.

Pharmaceutical compositions suitable for injection include sterile aqueous solutions or dispersions for the extemporaneous preparation of sterile injectable solutions or dispersion. For intravenous administration, suitable carriers include physiological saline, bacteriostatic water, Cremophor EL™ (BASF; Parsippany, N.J.) or phosphate buffered saline (PBS). The composition must be sterile and should be fluid so as to be administered using a syringe. Such compositions should be stable during manufacture and storage and must be preserved against contamination from microorganisms, such as bacteria and fungi. The carrier can be a dispersion medium containing, for example, water, polyol (such as glycerol, propylene glycol, and liquid polyethylene glycol), and other compatible, suitable mixtures. Various antibacterial and anti-fungal agents, for example, parabens, chlorobutanol, phenol, ascorbic acid, and thimerosal, can contain microorganism contamination. Isotonic agents such as sugars, polyalcohols, such as manitol, sorbitol, and sodium chloride can be included in the composition. Compositions that can delay absorption include agents such as aluminum monostearate and gelatin.

Sterile injectable solutions can be prepared by incorporating an iron complex in the required amount in an appropriate solvent with a single or combination of ingredients as required, followed by sterilization. Methods of preparation of sterile solids for the preparation of sterile injectable solutions include vacuum drying and freeze-drying to yield a solid containing the iron complex and any other desired ingredient.

Active compounds may be prepared with carriers that protect the compound against rapid elimination from the body, such as a controlled release formulation, including implants and microencapsulated delivery systems. Biodegradable or biocompatible polymers can be used, such as ethylene vinyl acetate, polyanhydrides, polyglycolic acid, collagen, polyorthoesters, and polylactic acid. Such materials can be obtained commercially from ALZA Corporation (Mountain View, Calif.) and NOVA Pharmaceuticals, Inc. (Lake Elsinore, Calif.), or prepared by one of skill in the art.

A preferred pharmaceutical composition for use in the methods described herein contains FCM as the active pharmaceutical ingredient (API) with about 28% weight to weight (m/m) of iron, equivalent to about 53% m/m iron (III)-hydroxide, about 37% m/m of ligand, ≦6% m/m of NaCl, and ≦10% m/m of water.

Agents or compositions described herein can also be used in combination with other therapeutic modalities, as described further below. Thus, in addition to the therapies described herein, one may also provide to the subject other therapies known to be efficacious for treatment of disease, disorder, or condition associated with IDA.

Oral Iron.

Iron preparations generally contain one of three iron salts: iron sulphate, iron gluconate, and iron fumurate. Iron carbohydrate complexes, as described above, can also be prepared to be taken orally.

Oral iron may be given as tablets or elixirs. Among the tablet preparations, there are non-enteric coated pills and enteric coated and prolonged-release formulations. Non-enteric coated iron tablets are most commonly used as initial treatment due to their lower cost. Delayed release and enteric-coated iron preparations have been advocated since they can be better tolerated than non-enteric coated tablets. But they may be less effective since they may contain less iron and their iron may not be released in the duodenum, where iron is absorbed. In fact, subjects who have been treated unsuccessfully with enteric-coated and prolonged-release iron preparations may respond well to the administration of non-enteric-coated ferrous salts.

Therapeutic Methods

Also provided is a process of treating a disease, disorder, or condition associated with iron deficiency anemia (IDA) in a subject in need of iron therapy. Such therapeutic treatment protocol can follow a determination of a hepcidin level in a subject. As described more fully above, based upon a determined level of hepcidin, a subject can be administered an oral iron formulation or an intravenous iron formulation, or a combination thereof. Administration of an oral iron supplement can be as described in Alleyne, 2008; Zhu 2010; and Aspuru 2011. Administration of an iron carbohydrate complex can be as described in U.S. Pat. No. 6,960,571, issued 1 Nov. 2005; U.S. Pat. No. 7,754,702, issued 13 Jul. 2010; International Patent Application No. WO 2007/081744, published 19 Jul. 2007; US Patent Application Publication No. 2010/0266644, published 21 Oct. 2010.

Generally, a safe and effective amount of an iron compound is, for example, that amount that would cause the desired therapeutic effect in a subject while minimizing undesired side effects. The dosage regimen can be determined by skilled clinicians, based on factors such as the exact nature of the condition being treated, the severity of the condition, the age and general physical condition of the subject, and so on.

According to the methods described herein, administration can be parenteral, pulmonary, oral, topical, intradermal, intramuscular, intraperitoneal, intravenous, subcutaneous, intranasal, epidural, ophthalmic, buccal, or rectal administration.

When used in the treatments described herein, a therapeutically effective amount of an iron compound can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to replenish iron stores in a subject.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Shamqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the above listed states, diseases, disorders, or conditions associated with IDA, as well as others, can benefit from the treatment methodologies described herein. Generally, treating a state, disease, disorder, or condition includes preventing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms.

The benefit to a subject to be treated is either statistically significant or at least perceptible to the patient or to the physician. Measures of efficacy of iron replacement therapy are generally based on measurement of iron-related parameters in blood or a blood component. The aim of treatment is usually to return both Hb and iron stores to normal levels. Thus, efficacy of iron replacement therapy can be interpreted in terms of the ability to normalize Hb levels and iron stores. The effectiveness of treatment with one or more single unit doses of iron carbohydrate complex, as described herein, can be demonstrated, for example, by improvements in ferritin and transferrin saturation, and in raising hemoglobin levels in anemic patients. Iron stores can be assessed by interpreting serum ferritin levels. TfS is frequently used, in addition, to diagnose absolute or functional iron deficiencies. In patients with iron deficiency, serum transferrin is elevated and will decrease following successful iron treatment. Treatment in accord with the methods described herein can be performed prior to, concurrent with, or after conventional treatment modalities for a disease, disorder, or condition associated with IDA.

An iron agent can be administered simultaneously or sequentially with another agent, such as an antibiotic, an antiinflammatory, or another agent. For example, an iron agent can be administered simultaneously with another agent, such as an antibiotic or an antiinflammatory. Simultaneous administration can occur through administration of separate compositions, each containing one or more of an iron agent, an antibiotic, an antiinflammatory, or another agent. Simultaneous administration can occur through administration of one composition containing two or more of a an iron agent (e.g., an iron carbohydrate complex), an antibiotic, an antiinflammatory, or another agent. An iron agent can be administered sequentially with an antibiotic, an antiinflammatory, or another agent. For example, an iron compound can be administered before or after administration of an antibiotic, an antiinflammatory, or another agent.

Subject in Need Thereof

Methods described herein can be performed on a subject in need thereof. A subject in need of a predictive or therapeutic method described herein can be a subject having, diagnosed with, suspected of having, or at risk for developing iron deficiency anemia or a disease, disorder, or condition associated with iron deficiency anemia.

The subject can be an animal subject, including a mammal, such as horses, cows, dogs, cats, sheep, pigs, mice, rats, monkeys, guinea pigs, and chickens, and humans. For example, the subject can be a human subject. As another example, the subject can be a human male subject. As another example, the subject can be a human female subject.

A determination of the need for treatment will typically be assessed by a history and physical exam consistent with the disease or condition at issue. Diagnosis of the various conditions treatable by the methods described herein is within the skill of the art. An iron agent described herein can be administered to a subject where there is a clinical need to deliver iron or in subjects with functional iron deficiency such as those on erythropoietin therapy. A determination of the need for treatment with parenteral iron is within the abilities of one skilled in the art. For example, need can be assessed by monitoring a subject's iron status. The diagnosis of iron deficiency can be based on appropriate laboratory tests, for example, haemoglobin (Hb), serum ferritin, serum iron, transferrin saturation (TfS), and hypochromic red cells.

A determination of the need for treatment with an iron agent can be also be determined through diagnosis of a subject suffering from a disease, disorder, or condition that is associated with iron deficiency anemia. For example, many chronic renal failure patients receiving erythropoietin will require an iron agent to maintain target iron levels. As another example, most hemodialysis patients will require administration of an iron agent due to dialysis-associated blood loss and resulting negative iron balance.

Monitoring frequency can depend upon the disease, disorder, or condition the subject is afflicted with or at risk for. For example, in a subject initiating erythropoietin therapy, iron indices are monitored monthly. As another example, in subject who have achieved target range Hb or are receiving intravenous iron therapy, TSAT and ferritin levels can be monitored every 3 months.

A subject's iron status can be indicative of an absolute or a functional iron deficiency, both of which can be treated with the compositions, predictive methods, and therapeutic methods described herein. An absolute iron deficiency occurs when an insufficient amount of iron is available to meet the body's requirements. The insufficiency may be due to inadequate iron intake, reduced bioavailability of dietary iron, increased utilization of iron, or chronic blood loss. Prolonged iron deficiency can lead to iron deficiency anemia—a microcytic, hypochromic anemia in which there are inadequate iron stores. Absolute iron deficiency is generally indicated where TSAT<20% and Ferritin<100 ng/mL.

Functional iron deficiency can occur where there is a failure to release iron rapidly enough to keep pace with the demands of the bone marrow for erythropoiesis, despite adequate total body iron stores. In these cases, ferritin levels may be normal or high, but the supply of iron to the erythron is limited, as shown by a low transferrin saturation and an increased number of microcytic, hypochromic erythrocytes. Functional iron deficiency can be characterized by the following characteristics: Inadequate hemoglobin response to erythropoietin; Serum ferritin may be normal or high; Transferrin saturation (TSAT) usually <20%; and/or reduced mean corpuscular volume (MCV) or mean corpuscular hemoglobin concentration (MCHC) in severe cases. Functional iron deficiency (i.e., iron stores are thought to be adequate but unavailable for iron delivery) is generally indicated where TSAT<20% and Ferritin>100 ng/mL.

Assessing the need for iron therapy can be according to the National Kidney Foundation's Kidney Disease Outcomes Quality Initiative. See NKF-K/DOQI, Clinical Practice Guidelines for Anemia of Chronic Kidney Disease (2000); Am J Kidney Dis (2001) 37(supp 1), S182-S238. The DOQI provides optimal clinical practices for the treatment of anemia in chronic renal failure. The DOQI guidelines specify intravenous iron treatment of kidney disease based on hemoglobin, transferrin saturation (TSAT), and ferritin levels.

Assessment of need for iron therapy can also be according to a subject's target iron level. For example, the target hemoglobin level of a subject can be selected as 11.0 g/dL to 12.0 g/dL (hematocrit approximately 33% to 36%). To achieve target hemoglobin with optimum erythropoietin doses, sufficient iron, can be provided to maintain TSAT≧20% and ferritin 100 ng/mL. In erythropoietin-treated subjects, if TSAT levels are below 20%, the likelihood that hemoglobin will rise or erythropoietin doses fall after iron administration is high. Achievement of target hemoglobin levels with optimum erythropoietin doses is associated with providing sufficient iron to maintain TSAT above 20%.

Iron therapy can be given to maintain target hemoglobin while preventing iron deficiency and also preventing iron overload. Adjusting dosage of iron to maintain target levels of hemoglobin, hematocrit, and laboratory parameters of iron storage is within the normal skill in the art. For example, where a subject is anemic or iron deficient, an iron agent can be administered when a patient has a ferritin<800, a TSAT<50, and/or a Hemoglobin<12. Iron overload can be avoided by withholding iron for TSAT>50% and/or ferritin>800 ng/mL.

Where a subject is not anemic or iron deficient but is in need of iron administration, for example a subject suffering from Restless Leg Syndrome, hemoglobin and TSAT levels are not necessarily relevant, while ferritin>800 can still provides a general cut off point for administration.

Administration

Agents and compositions described herein can be administered according to methods described herein in a variety of means known to the art.

Generally, total iron dosage will depend on the iron deficit of the subject. One skilled in the art can tailor the total iron dose required for a subject while avoiding iron overload, as overdosing with respect to the total required amount of iron should be avoided. An iron agent can be administered so as to deliver a calculated iron deficit dose. In some embodiments, an iron deficit dose can be calculated as follows:

If baseline TSAT<20% or Baseline Ferritin<50 ng/ml: Dose=Baseline weight (kg)×(15-Baseline Hgb [g/dL])×2.4+500 mg

OR

If baseline TSAT>20% and Baseline Ferritin>50 ng/mL: Dose=Baseline weight (kg)×(15-Baseline Hgb [g/dL])×2.4
(NOTE: Baseline Hgb equals the average of the last two central lab Hgb's)

Administration of an iron agent can occur as a single event or over a time course of treatment. For example, an iron agent can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Administration of an iron agent can be, for example, over pre-determined time intervals or in response to the appearance or reappearance of symptoms. For example, an iron agent can be re-administered upon recurrence of at least one symptom of a disease, disorder, or condition associated with IDA. As another example, an iron agent can be re-administered at some time period after the initial administration (e.g., after 4 days to 12 months).

Exemplary administration routes include, but are not limited to, parenteral, pulmonary, oral, topical, intradermal, intramuscular, intraperitoneal, intravenous, subcutaneous, intranasal, epidural, ophthalmic, buccal, or rectal administration.

Any route of delivery of an iron agent is acceptable so long as iron from the iron complex is released such that symptoms are treated. An iron agent can be administered parenterally, for example intravenously or intramuscularly. Intravenous administration can be delivered as a bolus or as an infusion. If applied as an infusion, an iron agent can be diluted with sterile saline (e.g., polynuclear iron (III)-hydroxide 4(R)-(poly-(1→4)-O-α-glucopyranosyl)-oxy-2(R),3(S),5(R),6-tetrahydroxy-hexanoate (“VIT-45”) 0.9% m/V NaCl or 500 mg iron in up to 250 mL NaCl). An iron agent can be intravenously injected as a bolus without dilution.

Delivery systems may include, for example, an infusion pump which may be used to administer an iron agent in a manner similar to that used for delivering insulin or chemotherapy to specific organs or tumors. Typically, using such a system, an iron agent can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve taste of the product; or improve shelf life of the product.

Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to a hepcidin screening assay or an iron agent (e.g., an intravenous iron carbohydrate complex or an oral iron supplement). Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline or sterile each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium, such as a floppy disc, mini-CD-ROM, CD-ROM, DVD-ROM, Zip disc, videotape, audio tape, and the like. Detailed instructions may not be physically associated with the kit; instead, a user may be directed to an Internet web site specified by the manufacturer or distributor of the kit.

Compositions and methods described herein utilizing molecular biology protocols can be according to a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1

### Subject Selection and Study Design

The following example describes hepcidin measurements obtained during a randomized, multi-center, controlled trial comparing the efficacy and safety of ferric carboxymaltose in subjects with iron deficiency anemia (IDA) (see generally, Szczech 2011).

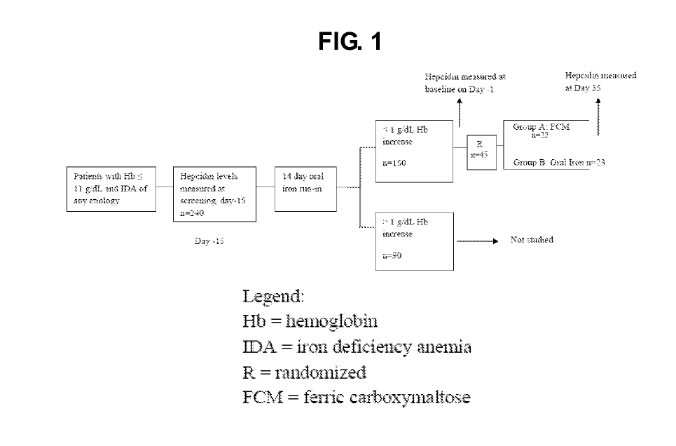

Eligibility for this trial included subjects≧18 years of age with hemoglobin≦11 g/dL and IDA of any etiology with ferritin≦100 ng/mL, or ≦300 ng/mL when transferrin saturation (TSAT) was ≦30%. The study design for this trial is illustrated in FIG. 1. Subjects who met the inclusion criteria received a 14-day course of oral iron (“oral iron run-in” ferrous sulfate 325 mg, three times daily for 14 days). Subjects who had an adequate response to the oral iron run-in (operationally defined as a hemoglobin increase of at least 1 g/dL in 14 days) were categorized as “responders”.

Those that did not have an adequate response to the oral iron run-in were classified as “non-responders” and randomized to treatment with either ferric carboxymaltose (2 injections of 750 mg given on Day 0 [day of randomization] and Day 7) from (Group A) or continuation of oral iron (Group B) for another 14 days. Ferric carboxymaltose (Group A) was administered at a dose of 15 mg/kg to a maximum 750 mg per dose administered intravenously on Day 0 and Day 7. Oral iron was given as ferrous sulfate 325 mg three times a day for an additional 14 days starting on Day 0.

Hemoglobin levels and markers of iron status were assessed up to 35 days and measures of safety and tolerability were assessed up to 120 days. Hemoglobin levels and markers of iron status were assessed at screening (Day 15), Day 1, and Day 35.

Hepcidin levels were analyzed from serum samples obtained at screening (Day 15) in an initial Analysis Group (I) of 44 subjects, 22 responders, and 22 non-responders. A hepcidin value of ≧20 ng/mL was identified as a cutoff for further study in the determination of predictive values for non-responsiveness to 14 day oral iron run-in in 240 subjects (Analysis Group II). Hepcidin levels were also analyzed at Day 1 and Day 35 in Analysis Group II of subjects who were then randomized to receive FCM or oral iron therapy.

The study showed that non-responsiveness to oral iron in subjects with iron deficiency anemia can be predicted from subjects' baseline hepcidin levels, which have superior positive predictive values compared to conventional transferrin saturation (TSAT) or ferritin levels.

### Example 2

### Hepcidin Screening Levels

The following example describes the selection of screening hepcidin levels for the prediction of non-response to oral iron. The subject groups assessed in this example included responders and non-responders to oral iron. An objective was to determine hepcidin levels that would predict non-response to oral iron.

Selection of screening (Day 15) hepcidin levels to predict if subjects would respond adequately to oral iron was based on an initial group (Analysis Group I) of 44 subjects (22 responders and 22 non-responders). The initial sample size for screening hepcidin levels in Analysis Group I was based on an estimated difference of mean 60±60 ng/mL between responders and non-responders (Zaritsky, 2009). Data from Analysis Group I was analyzed in a scatter plot (≧1 g/dL change in hemoglobin [y/n] vs. baseline hepcidin value) to identify a hepcidin value for further analysis (see e.g., FIG. 2). An additional 240 subjects (Analysis Group II) were evaluated to determine if hepcidin was a reliable predictor of non-response to oral iron (see e.g., Example 6).

Hepcidin levels were also assessed after the oral iron run-in (Day 1) and after treatment with intravenous (IV) iron or oral iron post-randomization (Day 35). Hepcidin levels were measured using a commercially available competitive enzyme-linked immunosorbent assay (C-ELISA). Samples were collected by the investigator at the site, stored by a contract research organization (Covance, Indianapolis, Ind.) and sent to a commercial laboratory (Intrinsic LifeSciences, La Jolla, Calif.) for analysis.

The data from all subjects in this sub-study, in which hepcidin measurements were available, were included for analysis. No imputation of missing data was performed. All statistical comparisons were made with a 0.05 Type I error for 2-sided tests. Tests for proportions were performed with Fisher's exact test or its generalized version for >2 categories. Mean differences were assessed with the t-test (i.e., 1-way analysis of variance for 2 groups), assuming equal variances. Correlations were estimated with Pearson's product-moment method. Logistic regression was used to evaluate the relationship between response to oral iron and screening values of hepcidin, ferritin, and transferrin saturation (TSAT) (see e.g., Example 3).

This example determined that 21 of 22 subjects with screening hepcidin values>20 ng/mL were non-responders. 20 ng/mL was selected as the cutoff point for further evaluation in the larger Analysis Group II (see e.g., Example 6).

### Example 3

### Screening Values for Hepcidin, Hemoglobin, Ferritin, Iron, Total Iron Binding Capacity (TIBC), and Transferrin Saturation (TSAT)

The following example describes the determination of potential screening levels of hepcidin, hemoglobin, ferritin, iron, total iron binding capacity (TIBC), and transferrin saturation (TSAT) in Analysis Group I.

Forty-four subjects (Analysis Group I, 22 non-responders and 22 responders) were studied in the initial analysis. Screening values for hepcidin, hemoglobin, ferritin, iron, total iron binding capacity (TIBC), and transferrin saturation (TSAT) were assessed. A comparison of non-responders to responders for screening hepcidin and iron indices are detailed in Table 1. For this initial analysis group, screening hemoglobin levels were higher in non-responders vs. responders (p=0.0012). Screening hepcidin levels were significantly higher in non-responders vs. responders (33.2 vs. 8.7 ng/mL, p<0.004). Mean ferritin levels were also significantly higher in the non-responders vs. responders (26.4 vs. 7.2, p=0.03). Differences in screening TSAT levels did not reach statistical significance.

TABLE 1
Screening Hepcidin and Iron Indices of Non-
responders and Responders to 14 Day Oral Iron Run-In
(Analysis Group I)

| | | Non-Responder | | |
|---|---|---|---|---|
| | | (Hb Δ | Responder | |
| Screening | Descriptive | <1 g/dL) | (Hb Δ ≧1 g/dL) | |
| Analyte | Statistic | (N = 22) | (N = 22) | p-value |
| Hepcidin | Mean (SD) | 33.2 (36.6) | 8.7 (7.1) | 0.0036 |
| | Median | 20.8 | 7.8 | |
| | Min, Max | 0.0, 139.3 | 0.0, 20.2 | |
| Hemoglobin | Mean (SD) | 10.1 (0.8) | 9.1 (1.1) | 0.0012 |
| | Median | 10.1 | 9.0 | |
| | Min, Max | 7.6, 11.6 | 7.3, 11.1 | |
| Ferritin | Mean (SD) | 26.4 (39.7) | 7.2 (9.4) | 0.0326 |
| | Median | 10.7 | 3.9 | |
| | Min, Max | 2.2, 176.7 | 1.0, 38.2 | |
| Iron | Mean (SD) | 45.3 (35.3) | 30.8 (47.3) | 0.2567 |
| | Median | 28.0 | 15.5 | |
| | Min, Max | 8, 144 | 8, 234 | |
| TIBC | Mean (SD) | 326.5 (64.4) | 361.3 (45.8) | 0.0453 |
| | Median | 316.5 | 375.0 | |
| | Min, Max | 200, 461 | 269, 444 | |
| TSAT | Mean (SD) | 14.3 (10.6) | 8.5 (12.0) | 0.0950 |
| | Median | 8.5 | 4.0 | |
| | Min, Max | 2, 42 | 2, 59 | |

Further, in a previous study that investigated 44 subjects with mild to moderate kidney disease being treated with erythropoietin, hepcidin levels were found to be positively correlated with ferritin levels (Ashby, 2009). Similarly, hepcidin was also positively correlated with ferritin (Pearson R=0.81, p<0.0001) in the sample. Serum ferritin levels are affected by inflammation, in which hepcidin internalizes and degrades the ferroportin transport molecule, entrapping ferritin-bound iron with macrophages (Nemeth, 2004). This shift to ferritin-bound iron is reflected in a corresponding increase in serum ferritin, thus termed an “acute phase reactant” to inflammation. Previous studies have shown that only when ferritins are in excess of 30 ng/mL does this assay have greater than 92% sensitivity for identifying subjects with absolute iron deficiency (Mast, 1998).

This study showed screening hemoglobin levels were higher in non-responders than in responders. Screening hepcidin levels were significantly higher in non-responders than in responders. Mean ferritin levels were also significantly higher in the non-responders than in responders. TSAT levels in non-responders vs. responders was not statistically significant.

### Example 4

### Screening Hemoglobin Vs. Screening Hepcidin with Response to Oral Iron

The following example describes the determination and comparison of hepcidin levels in 240 subjects with response to oral iron. Hepcidin and other iron indices were measured and compared. The objective of the study was to determine the baseline characteristics of the subject population and to compare levels of hepcidin, hemoglobin, and iron indices in responders and non-responders after a 14 day treatment of oral iron.

As shown in Example 2, a scatter plot of screening hemoglobin vs. screening hepcidin with response to oral iron for Analysis Group I is depicted in FIG. 2. This scatter plot indicated that 21 of 22 subjects with screening hepcidin values>20 ng/mL were non-responders, and this cutoff point was selected for further evaluation in the larger Analysis Group II. The subjects with a hepcidin value less than 20 ng/mL were grouped as responders.

Based on Analysis Group I, the predictive value of hepcidin level>20 ng/mL to predict non-responsiveness (increase in hemoglobin of ≦1 g/dL) to oral iron was tested in a larger analysis group (i.e., Analysis Group II) of 240 subjects (150 non-responders and 90 responders, also including the forty-four subjects in the initial Analysis Group I). Demographic and baseline characteristics of Analysis Group II are summarized in Table 2. Two hundred twenty-six (>90%) of the subjects were female. 108 (43%) and 35 (14%) had dysfunctional uterine bleeding (DUB) and gastrointestinal bleeding, respectively.

TABLE 2
Demographic and Baseline Characteristics for Analysis
Group II

| | Non-Responder | Responder | |
|---|---|---|---|
| | (Hb Δ <1 g/dL) | (Hb Δ ≧1 g/dL) | |
| Characteristic | (N = 150) | (N = 90) | p-value^{a} |
| Sex, n (%) | | | 0.1550 |
| Female | 144 (96.0) | 82 (91.1) | |
| Male | 6 (4.0) | 8 (8.9) | |
| Age, years | | | 0.1867 |
| Mean (SD) | 45.3 (17.5) | 42.4 (15.4) | |
| Median | 42.0 | 41.0 | |
| Race, n (%) | | | 0.2144 |
| African-American | 47 (31.3) | 29 (32.2) | |
| Caucasian | 48 (32.0) | 37 (41.1) | |
| Other | 55 (36.7) | 24 (26.7) | |
| Etiology, n (%) | | | 0.7724 |
| CKD | 2 (1.3) | 0 | |
| GI | 23 (15.3) | 12 (13.3) | |
| DUB | 68 (45.3) | 40 (44.4) | |
| Other | 57 (38.0) | 38 (42.2) | |
| Hx of iron therapy, n (%) | 85 (56.7) | 62 (68.9) | 0.0751 |
| Hx of iron intolerance, n | 2 (1.3) | 2 (2.2) | 0.6319 |
| (%) | | | |

From Fisher's exact test (or its generalization) for qualitative variables and from t-test for continuous variables.
CKD: chronic kidney disease
GI: gastrointestinal bleeding
DUB: dysfunctional uterine bleeding

A comparison for screening hemoglobin, hepcidin and iron indices of non-responders to responders in Analysis Group II is provided in Table 3. Screening hemoglobin was higher in non-responders vs. responders, (10.1 vs. 9.3 g/dL, p<0.0001). Subjects within Analysis Group II reflected greater hemoglobin changes in responders vs. non-responders after the 14 day oral iron run-in (1.8±0.6 vs. 0.2±0.4 g/dL, respectively). Screening hepcidin levels were significantly higher in non-responders vs. responders (38.4 vs. 11.3 ng/mL, p=0.0002). Screening ferritin values were also higher in non-responders than in responders (31.9 vs. 12.2 ng/mL, p<0.03).

TABLE 3
Screening Hepcidin and Iron Indices of Non-responders
and Responders to 14 Day Oral Iron Run-In (Analysis Group II)

| | | Non-Responder | | |
|---|---|---|---|---|
| | | (Hb Δ < | Responder | |
| | Descriptive | 1 g/dL) | (Hb Δ ≧ 1 g/dL) | |
| Analyte | Statistic | (N = 150) | (N = 90) | p-value^{a} |
| Hepcidin | Mean (SD) | 38.4 (66.7) | 11.3 (19.0) | 0.0002 |
| | Median | 14.70 | 6.40 | |
| | Min, Max | 0.0, 540.7 | 0.0, 127.5 | |
| Hemoglobin | Mean (SD) | 10.1 (1.0) | 9.3 (1.2) | <0.0001 |
| | Median | 10.25 | 9.40 | |
| | Min, Max | 5.6, 12.0 | 5.5, 11.8 | |
| Ferritin | Mean (SD) | 31.9 (82.9) | 12.2 (16.2) | 0.0270 |
| | Median | 9.85 | 6.20 | |
| | Min, Max | 0.8, 881.4 | 0.9, 101.0 | |
| Iron | Mean (SD) | 40.0 (28.5) | 36.3 (51.6) | 0.4761 |
| | Median | 29.5 | 22.5 | |
| | Min, Max | 8, 152 | 7, 399 | |
| TIBC | Mean (SD) | 339.9 (72.2) | 366.4 (58.1) | 0.0035 |
| | Median | 339.5 | 374.5 | |
| | Min, Max | 175, 518 | 174, 525 | |
| TSAT | Mean (SD) | 12.6 (9.3) | 10.0 (13.0) | 0.0732 |
| | Median | 9.0 | 6.0 | |
| | Min, Max | 2, 48 | 2, 96 | |

from 1-way ANOVA

The study showed hemoglobin levels were higher in non-responders than responders after the 14 day oral iron run-in. Further, greater hemoglobin changes were observed in responders than non-responders after the 14 day iron treatment when compared to baseline hemoglobin levels. Hepcidin levels were significantly higher in non-responders than responders. Ferritin values were also higher in non-responders than responders.

### Example 5

### Correlation of Hepcidin and Other Analytes

The following example describes the correlation values of hepcidin with other analytes.

The correlation between screening hepcidin levels and the other screening analytes is shown in Table 4. The strongest positive correlation in non-responders between screening hepcidin and screening iron indices was with ferritin (Pearson R=0.81, p<0.0001). A positive correlation between screening hepcidin and screening ferritin was also observed in the responders (Pearson R=0.59, p<0.0001).

TABLE 4
Pearson Correlation of Screening Hepcidin with
Other Screening Analytes

| | | | Responder | |
|---|---|---|---|---|
| | | Non-Responder | (Hb Δ | |
| Screening | Descriptive | (Hb Δ <1 g/dL) | ≧1 g/dL) | Total |
| Analyte | Statistic | (N = 150) | (N = 90) | (N = 240) |
| Age | Pearson R | 0.31 | −0.16 | 0.24 |
| | P-value | 0.0001 | 0.1307 | 0.0002 |
| Hemoglobin | Pearson R | 0.01 | 0.24 | 0.12 |
| | P-value | 0.9486 | 0.0241 | 0.0697 |
| Ferritin | Pearson R | 0.81 | 0.59 | 0.81 |
| | P-value | <0.0001 | <0.0001 | <0.0001 |
| Iron | Pearson R | 0.17 | 0.04 | 0.11 |
| | P-value | 0.0330 | 0.7422 | 0.0807 |
| TIBC | Pearson R | −0.49 | −0.27 | −0.46 |
| | P-value | <0.0001 | 0.0098 | <0.0001 |
| TSAT | Pearson R | 0.35 | 0.08 | 0.27 |
| | P-value | <0.0001 | 0.4763 | <0.0001 |

NOTE:
The percent of variability in hepcidin explained by a screening analyte can be estimated by R × R × 100 (e.g., the percent of variability in hepcidin explained by age is 0.24 × 0.24 × 100 = 5.8% for all subjects)

This study showed a positive correlation between hepcidin and ferritin in both non-responder and responder groups.

### Example 6

### Prediction of Non-Responsiveness to Oral Iron Therapy Using Screening Values of Hepcidin

The following example describes the use of screening values of hepcidin to predict non-responsiveness to oral iron therapy. The sensitivity and specificity of using hepcidin, ferritin, and TSAT values for prediction of non-responsiveness to oral iron therapy were evaluated.

Utilizing the hepcidin criterion of >20 ng/mL (see e.g., Table 5) a sensitivity of 41.3% (62 of 150), specificity of 84.4% (76 of 90), and a positive predictive value (PPV) of 81.6% (62 of 76) was determined for non-responsiveness to oral iron. While ferritin<30 ng/mL or TSAT<15% had greater sensitivity (77.3% and 64.7% respectively), the PPVs of ferritin and TSAT (59.2% and 55%, respectively) were inferior to the PPVs of hepcidin levels (81.6%).

TABLE 5
Predictive Values for Screening Hepcidin Levels and Iron
Indices for Non-Responsiveness to Oral Iron Therapy

| | Predicted | Observed | Number | | | | |
|---|---|---|---|---|---|---|---|
| Criteria | Response | Response | Subjects | Sensitivity | Specificity | PPV | NPV |
| Hepcidin > 20 | No | No | 62 | 62/150 | 76/90 | 62/76 | 76/164 |
| | No | Yes | 14 | (41.3) | (84.4) | (81.6) | (46.3) |
| | Yes | No | 88 | | | | |
| | Yes | Yes | 76 | | | | |
| Ferritin < 30 | No | No | 116 | 116/150 | 10/90 | 116/196 | 10/44 |
| | No | Yes | 80 | (77.3) | (11.1) | (59.2) | (22.7) |
| | Yes | No | 34 | | | | |
| | Yes | Yes | 10 | | | | |
| TSAT < 15 | No | No | 97 | 97/150 | 13/90 | 97/174 | 13/66 |
| | No | Yes | 77 | (64.7) | (14.4) | (55.7) | (19.7) |
| | Yes | No | 53 | | | | |
| | Yes | Yes | 13 | | | | |

Sensitivity = 100* (# non-responders correctly predicted)/(# observed non-responders)
Specificity = 100* (# responders correctly predicted)/(# observed responders)
PPV = 100* (# non-responders correctly predicted)/(# predicted non-responders)
NPV = 100* (# responders correctly predicted)/(# predicted responders)

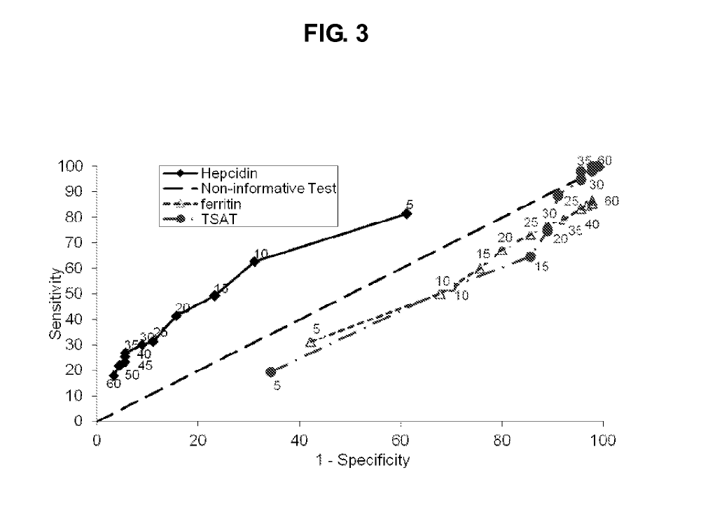

A receiver operator curve (ROC) was generated to determine the informativeness of the analyte levels (see e.g., FIG. 3). FIG. 3 illustrates the receiver operator characteristic (ROC) curve using hepcidin, ferritin, and TSAT criterion. Hepcidin was superior to both ferritin and TSAT (both of which fell below the non-informative line). Furthermore, sensitivity, specificity, and positive predicting values over a range of hepcidin thresholds are shown in Table 6.

TABLE 6
Predictive Values for Selected Hepcidin Criteria

| Hepcidin | | | | |
|---|---|---|---|---|
| Criteria | Sensitivity | Specificity | PPV | NPV |
| >5 | 81.3 | 38.9 | 68.9 | 55.6 |
| >10 | 62.7 | 68.9 | 77.0 | 52.5 |
| >15 | 49.3 | 76.7 | 77.9 | 47.6 |
| >20 | 41.3 | 84.4 | 81.6 | 46.3 |
| >25 | 31.3 | 88.9 | 82.5 | 43.7 |
| >30 | 30.0 | 91.1 | 84.9 | 43.9 |
| >35 | 26.7 | 94.4 | 88.9 | 43.6 |
| >40 | 25.3 | 94.4 | 88.4 | 43.1 |
| >45 | 23.3 | 94.4 | 87.5 | 42.5 |
| >50 | 22.0 | 95.6 | 89.2 | 42.4 |
| >55 | 19.3 | 96.7 | 90.6 | 41.8 |
| >60 | 18.0 | 96.7 | 90.0 | 41.4 |
| >65 | 16.7 | 96.7 | 89.3 | 41.0 |
| >70 | 16.7 | 97.8 | 92.6 | 41.3 |

Analyses for sensitivity, specificity, and positive predictive value were also performed for ferritin (Table 7) and TSAT levels (Table 8). Within the range of thresholds, a hepcidin criteria of >20 ng/mL had a superior positive predictive value for predicting hemoglobin non-responsiveness to oral iron therapy compared to ferritin or TSAT.

TABLE 7
Predictive Values for Selected Ferritin Criteria

| Ferritin | | | | |
|---|---|---|---|---|
| Criteria | Sensitivity | Specificity | PPV | NPV |
| <5 | 31.3 | 57.8 | 55.3 | 33.5 |
| <10 | 50.0 | 32.2 | 55.1 | 27.9 |
| <15 | 60.0 | 24.4 | 57.0 | 26.8 |
| <20 | 67.3 | 20.0 | 58.4 | 26.9 |
| <25 | 79.3 | 14.4 | 58.8 | 24.5 |
| <30 | 77.3 | 11.1 | 59.2 | 22.7 |
| <35 | 79.3 | 7.8 | 58.9 | 18.4 |
| <40 | 83.3 | 4.4 | 59.2 | 13.8 |
| <45 | 84.7 | 3.3 | 59.3 | 11.5 |
| <50 | 85.3 | 2.2 | 59.3 | 8.3 |
| <55 | 86.0 | 2.2 | 59.4 | 8.7 |
| <60 | 86.7 | 2.2 | 59.6 | 9.1 |
| <65 | 87.3 | 2.2 | 59.8 | 9.5 |
| <70 | 88.7 | 2.2 | 60.2 | 10.5 |

TABLE 8
Predictive Values for Selected TSAT Criteria

| TSAT | | | | |
|---|---|---|---|---|
| Criteria | Sensitivity | Specificity | PPV | NPV |
| <5 | 19.3 | 65.6 | 48.3 | 32.8 |
| <10 | 52.7 | 30.0 | 55.6 | 27.6 |
| <15 | 64.7 | 14.4 | 55.7 | 19.7 |
| <20 | 74.7 | 11.1 | 58.3 | 20.8 |
| <25 | 88.7 | 8.9 | 61.9 | 32.0 |
| <30 | 94.7 | 4.4 | 62.3 | 33.3 |
| <35 | 98.0 | 4.4 | 63.1 | 57.1 |
| <40 | 98.0 | 2.2 | 62.6 | 40.0 |
| <45 | 99.3 | 2.2 | 62.9 | 66.7 |
| <50 | 100.0 | 2.2 | 63.0 | 100.0 |
| <55 | 100.0 | 2.2 | 63.0 | 100.0 |
| <60 | 100.0 | 1.1 | 62.8 | 100.0 |
| <65 | 100.0 | 1.1 | 62.8 | 100.0 |
| <70 | 100.0 | 1.1 | 62.8 | 100.0 |

These analyses demonstrated significant differences in screening mean hepcidin levels between subjects who are subsequently identified to be “non-responders” to oral iron (i.e., demonstrate less than 1 g/dL increase in hemoglobin over 14 days) vs. responders. A hepcidin level of >20 ng/mL was determined to show a positive predictive value of 81.6% for non-responsiveness to oral iron therapy.

The study shows that non-responsiveness to oral iron in subjects with iron deficiency anemia can be predicted from subjects' baseline hepcidin levels, which have superior positive predictive values compared to transferrin saturation (TSAT) or ferritin levels.

### Example 7

### Effect of FCM of Oral Iron Therapy on Non-Responders

The following example describes the randomized study of ferric carboxymaltose (FCM) vs. oral iron therapy on non-responders. Baseline (Day 1) and post-treatment (Day 35) levels of hepcidin and hemoglobin were compared.

The effect of FCM or oral iron therapy on hepcidin and hemoglobin levels from baseline (Day 1) to Day 35 was investigated in 45 of the 240 non-responders to the oral iron run-in with these data and is summarized in Table 9.

Subjects randomized to Group A (ferric carboxymaltose) or Group B (oral iron) had similar baseline mean hepcidin levels, (39.15 vs. 32.03 ng/mL, respectively). A greater mean change in hepcidin between Day 1 and Day 35 was observed in subjects randomized to ferric carboxymaltose vs. subjects randomized to oral iron therapy. In the ferric carboxymaltose group, mean hepcidin levels increased from a baseline of 39.15 to 160.51 ng/mL at Day 35 with a change in mean hepcidin of 121.35 ng/mL. Subjects randomized to oral iron had no significant change in hepcidin by Day 35.

Mean hemoglobin changes were significantly greater in subjects randomized to ferric carboxymaltose compared to oral iron therapy (1.7 vs. 0.6 g/dL, p=0.0025, respectively). Non-responders to the oral iron run-in (in whom hepcidin values were measured) were subsequently randomized to ferric carboxymaltose (FCM) had a greater percentage of subjects with a hemoglobin increase of ≧1 g/dL compared to oral iron (65.3 vs. 20.8%, p<0.0001, respectively) and mean Hemoglobin increases of 1.7±1.3 vs. 0.6±0.9 gm/dL for those randomized to FCM vs. oral iron, respectively. Similar results were also demonstrated for hemoglobin change ≧2 g/dL (37.5% vs. 5.2% for FCM and oral iron, respectively).

TABLE 9
Change in hepcidin and hemoglobin levels
from baseline (Day 1) to Day 35 for non-responders to
the oral iron run-in

| | Group A: FCM | Group B: Oral iron | |
|---|---|---|---|
| Analyte | (N = 22) | (N = 23) | P Value |
| Hepcidin at | 39.2 (92.236) | 32.0 (44.314) | 0.7409 |
| baseline (Day 1) | 2.5 | 19.6 | |
| | 0.0, 425.5 | 0.0, 171.2 | |
| Hepcidin on Day | 160.5 (181.543) | 29.0 (53.894) | 0.0018 |
| 35 | 112.6 | 10.5 | |
| | 0.0, 724.4 | 0.0, 224.5 | |
| Hepcidin change | 121.35 (115.977) | −3.02 (41.793) | <0.0001 |
| to Day 35 | 91.9 | −6.0 | |
| | 0.0, 511.5 | −62.5, 168.7 | |
| Hemoglobin at | 10.3 (1.1) | 10.4 (1.2) | 0.8142 |
| baseline (Day 1) | 10.4 | 10.7 | |
| | 7.9, 11.9 | 7.3, 12.5 | |
| Hemoglobin on | 12.0 (1.4) | 11.0 (1.3) | 0.0186 |
| Day 35 | 12.2 | 10.9 | |
| | 9.8, 14.6 | 8.7, 13.5 | |
| Hemoglobin | 1.7 (1.3) | 0.6 (0.9) | 0.0025 |
| change to Day 35 | 1.7 | 0.5 | |
| | −0.9, 3.4 | −1.3, 2.8 | |

Values shown are mean (SD), median, and minimum/maximum

The study showed a change of hepcidin from baseline (Day 1) to Day 35 in subjects randomized between intravenous iron (ferric carboxymaltose) and oral iron therapy. A significant increase was shown in hepcidin with ferric carboxymaltose therapy but no significant change in hepcidin with oral iron therapy was found. An increase in hepcidin would be expected if oral iron therapy resulted in increased iron stores. Because an increase did not occur in the oral iron group, this demonstrated an inferior increment in iron stores when compared to FCM therapy (Qunibi, 2011). Ferric carboxymaltose also increased hemoglobin levels significantly when compared to oral iron therapy. The markedly superior response rates to FCM vs. oral iron therapy (65.3 vs. 20.8%, respectively) confirmed the efficacy of this anemia management strategy even in subjects who have previously been demonstrated to be non-responsive to oral iron. Screening hepcidin levels can predict subjects who are non-responders, thus making a clinical trial of oral iron therapy both undesirable and unnecessary. Finally, traditional management of IDA with the use of oral iron therapy can be misleading, since neither ferritin or TSAT have desirable PPVs. In subjects with IDA, hepcidin levels were significantly higher and were better predictors when compared to transferrin saturation or ferritin levels, for non-responders after a course of oral iron. Furthermore, non-responsiveness to oral iron therapy does not rule out iron deficiency in subjects with anemia since IV FCM therapy produced hemoglobin responses in two thirds of subjects who had no response to a trial of oral iron therapy.

### REFERENCES

1. Clark S F. Iron deficiency anemia. Nutr Clin Pract 2008; 23:128-41.

2. Weiss G, Goodnough L T. Anemia of chronic disease. N Engl J Med 2005; 352:1011-23.

3. Coyne D W. Hepcidin: clinical utility as a diagnostic tool and therapeutic target. Kidney Int 2011; 80:240-4.

4. Nemeth E, Tuttle M S, Powelson J, et al. Hepcidin regulates cellular iron efflux by binding to ferroportin and inducing its internalization. Science 2004; 306:2090-3.

5. Ganz T, Nemeth E. Iron imports. IV. Hepcidin and regulation of body iron metabolism. Am J Physiol Gastrointest Liver Physiol 2006; 290:6199-203.

6. Ganz T, Nemeth E. The hepcidin-ferroportin system as a therapeutic target in anemias and iron overload disorders. Hematology Am Soc Hematol Educ Program 2011; 2011:538-42.

7. Goodnough L T, Nemeth E, Ganz T. Detection, evaluation, and management of iron-restricted erythropoiesis. Blood 2010; 116:4754-61.

8. Freireich E J, Miller A, Emerson C P, Ross J F. The effect of inflammation on the utilization of erythrocyte and transferrin bound radioiron for red cell production. Blood 1957; 12:972-83.

9. Ganz T, Nemeth E. Hepcidin and disorders of iron metabolism. Annu Rev Med 2011; 62:347-60.

10. Goodnough L T. Iron deficiency syndromes and iron-restricted erythropoiesis. Transfusion. Epub Dec. 29, 2011.

11. Szczech L A, Bregman D B, Harrington R A, Morris D, Butcher A, Koch T A, Goodnough L T, Onken J E. Comparison of high dose carboxymaltose to oral or IV iron in subjects with iron deficiency anemia not suitable for oral iron. Amer Soc Nephrol 2011; 22:405A.

12. Zaritsky J, Young B, Wang H J, et al. Hepcidin—a potential novel biomarker for iron status in chronic kidney disease. Clin J Am Soc Nephrol 2009; 4:1051-6.

13. Fleming R E, Ponka P. Iron overload in human disease. N Engl J Med 2012; 366:348-59.

14. Ashby D R, Gale D P, Busbridge M, et al. Plasma hepcidin levels are elevated but responsive to erythropoietin therapy in renal disease. Kidney Int 2009; 75:976-81.

15. Mast A E, Blinder M A, Gronowski A M, Chumley C, Scott M G. Clinical utility of the soluble transferrin receptor and comparison with serum ferritin in several populations. Clin Chem 1998; 44:45-51.

16. Qunibi W Y, Martinez C, Smith M, Benjamin J, Mangione A, Roger S D. A randomized controlled trial comparing intravenous ferric carboxymaltose with oral iron for treatment of iron deficiency anaemia of non-dialysis-dependent chronic kidney disease subjects. Nephrol Dial Transplant 2011; 26:1599-607.

17. Wish et al., Assessing Iron Status: Beyond Serum Ferritin and Transferring Saturation, Clin J Am Soc Nephrol, 2006; 1:S4-S8.

18. Ganz et al., Immunoassay for Hepcidin, Blood, 2008, pp. 4292-4297, Vol. 112.

19. Alleyne et al., Individualized treatment for iron deficiency anemia in adults, Am J Med, 2008, pp. 943-948, Vol. 121, No. 11.

20. Zhu et al., Evaluation and treatment of Iron deficiency Anemia: A Gastroenterological Perspective, 2010, pp. 548-559, Vol. 55.

21. Aspuru et al., Optimal management of iron deficiency anemia due to poor dietary intake, Int J Gen Med, 2011, pp. 741-750, Vol. 4.

22. Van Wyck et al. (2004) J Am Soc Nephrol 15, S91-S92.

23. Spinowitz et al. (2005) Kidney Intl 68, 1801-1807.

24. Landry et al. (2005) Am J Nephrol 25, 400-410, 408.

25. Pasricha et al. 2011 Haematologica 96(8), 1099-1105.

What is claimed is:

1. A method of determining response to iron therapy for treatment of iron deficiency anemia or a disease, disorder, or condition associated with iron deficiency anemia in a subject comprising:
(a) determining a level of hepcidin in a biological sample of a subject in need of iron therapy; and
(b) at least one of
(i) correlating a level of hepcidin equal to or greater than a predetermined hepcidin level with reduced responsiveness of the subject to oral iron therapy; or
(ii) correlating a level of hepcidin less than the predetermined hepcidin level with at least adequate responsiveness of the subject to oral iron therapy.

2. A method of treating iron deficiency anemia or a disease, disorder, or condition associated with iron deficiency anemia, comprising:
(a) determining a level of hepcidin in a biological sample of a subject in need of iron therapy;
(b) at least one of
(i) correlating a level of hepcidin equal to or greater than a predetermined hepcidin level with reduced responsiveness of the subject to oral iron therapy; or
(ii) correlating a level of hepcidin less than the predetermined hepcidin level with at least adequate responsiveness of the subject to oral iron therapy; and
(c) administering a composition comprising iron, wherein administering the composition comprises
(i) intravenously administering a first iron composition to the subject if the level of hepcidin is equal to or greater than the predetermined hepcidin level; or
(ii) orally administering a second iron composition to the subject if the level of hepcidin is less than the predetermined hepcidin level.

3. The method of claim 2, wherein the first iron composition comprises an iron carbohydrate complex.

4. The method of any one of claims 2-3, wherein the first iron composition comprises one or more of an iron carboxymaltose, iron dextran; sodium ferric gluconate complex in sucrose; ferumoxytol, iron sucrose; iron gluconate; iron dextrin; polymaltose; iron sucrose; iron saccharate complex; iron pyrophosphate; or iron sorbitol.

5. The method of claim 3, wherein the first iron carbohydrate complex comprises an iron carboxymaltose.

6. The method of any one of claims 2-5, wherein the second iron composition comprises one or more of an iron (II) sulfate; ferrous sulfate; ferrous fumarate; heme iron polypeptide; ferrous glycine sulfate; iron pyrophosphate; or an iron carbohydrate complex.

7. The method of any one of claims 1-6, wherein the disease, disorder, or condition associated with iron deficiency anemia comprises at least one of: chronic blood loss; acute blood loss; pregnancy; a post-partum time period; a peripartum time period; dysfunctional uterine bleeding; heavy uterine bleeding; chronic recurrent hemoptysis; chronic internal bleeding; gastrointestinal bleeding; parasitic infections; chronic kidney disease; dialysis; surgery or acute trauma; use of erythropoiesis stimulating agents; insufficient dietary intake and absorption of iron; iron loss from intestinal bleeding; parasitic worms; bleeding ulcer; gastric ulcers; duodenal ulcers; gastrointestinal cancer; colon polyp; urinary tract bleeding; blood loss from injury, surgery, or frequent blood drawing; gastric bypass; disease of the intestine; Crohn's disease; celiac disease; malabsorption; or iron deficiency anemia of unknown etiology.

8. The method of any one of claims 1-7, wherein the iron deficiency anemia or the disease, disorder, or condition associated with iron deficiency anemia comprises inflammation.

9. The method of any one of claims 1-8, wherein reduced responsiveness of the subject to oral iron therapy comprises less than about 1 g/dL increase in hemoglobin over about two weeks of oral iron treatment.

10. The method of any one of claims 1-9, wherein an at least adequate responsiveness of the subject to oral iron therapy comprises more than about 1 g/dL increase in hemoglobin over about two weeks of oral iron treatment.

11. The method of any one of claims 1-9, wherein determining a level of hepcidin in a subject comprises a hepcidin immunoassay or a hepcidin mass spectrophotometry assay.

12. The method of any one of claims 1-11, wherein the predetermined hepcidin level is about 10 ng/mL, about 11 ng/mL, about 12 ng/mL, about 13 ng/mL, about 14 ng/mL, about 15 ng/mL, about 16 ng/mL, about 20 ng/mL, about 21 ng/mL, about 22 ng/mL, about 23 ng/mL, about 24 ng/mL, about 25 ng/mL, about 30 ng/mL, about 40 ng/mL, about 50 ng/mL, about 60 ng/mL, about 70 ng/mL, about 80 ng/mL, about 90 ng/mL, about 100 ng/mL, about 110 ng/mL, about 120 ng/mL, about 130 ng/mL, about 140 ng/mL, about 150 ng/mL, about 160 ng/mL, about 170 ng/mL, about 180 ng/mL, about 190 ng/mL, about 200 ng/mL, about 210 ng/mL, about 220 ng/mL, about 230 ng/mL, about 240 ng/mL, about 250 ng/mL, about 260 ng/mL, about 270 ng/mL, about 280 ng/mL, about 290 ng/mL, about 300 ng/mL, about 310 ng/mL, about 320 ng/mL, about 330 ng/mL, about 340 ng/mL, about 350 ng/mL, about 360 ng/mL, or about 370 ng/mL.

13. The method of any one of claims 1-11, wherein the predetermined hepcidin level is about 10 ng/ml.

14. The method of any one of claims 1-11, wherein the predetermined hepcidin level is about 15 ng/ml.

15. The method of any one of claims 1-11, wherein the predetermined hepcidin level is about 20 ng/ml.

16. The method of any one of claims 1-15, wherein the subject is a mammal.

17. The method of any one of claims 1-16, wherein the subject is a human.

18. The method of any one of claims 1-17, wherein the biological sample comprises at least one selected from the group consisting of urine, whole blood, or a blood component.

19. The method of any one of claims 1-18, wherein the biological sample comprises serum.
